(19) United States
(12) Patent Application Publication
(10) Pub. No.: US 2007/0066921 A1
(43) Pub. Date: Mar. 22, 2007

(54) Cushioning pad with water-repellent skin

(76) Inventor: Richard J. Garelick, Minneapolis, MN (US)

Correspondence Address: Marvin Jacobson;Jacobson and Johnson, Suite 285, One West Water Street, St. Paul, MN, 55107-2080, US

(21) Appl. No.: 11/208,309

(22) Filed: Aug. 22, 2005

(51) Int. Cl. A61F 5/00

(52) U.S. Cl. 602/6

(57) ABSTRACT

A cushioning pad of open-cell polyurethane material has a thin water-repellent skin bonded directly onto the surface of the pad without any intervening adhesive layer by spraying a two-component combination of resin and Isocyanate in situ directly onto the pad.

### FIELD OF THE INVENTION

This invention is aimed at providing a cushioning pad such as used on a seat or a bed having a thin water-repellent skin bonded directly onto a surface of the pad without any intervening adhesive layer and a method for producing an article of this nature. More specifically, the invention is directed toward a product of the nature described in which the cushioning pad is an open cell foam cushioning material.

### DESCRIPTION OF THE PRIOR ART

There are a number of commercially available seat cushions or the like which have a padding of open cell foam such as polyurethane foam enclosed in or covered by a relatively thin layer of water-repellent material where the covering is not bonded directly onto the surface of the foam padding. Also there are foam pads or cushions having a water-repellent skin which is attached to the pad by an intervening layer of adhesive.

There is also an open-cell polyurethane pad with a bonded skin which is produced by first spraying the interior of the shell of a mold with a resin and then inserting a polyurethane cushioning material into the mold and then closing the mold shell and applying suitable pressure and temperature so that the thin layer of resin adheres directly onto the surface of the foam material when the mold is opened. This process for making the molded product as described hereinabove is a process used by or originated by BASF Corporation located in Wyandotte, Mich. and is described and shown in a CD which is available from that company.

### SUMMARY OF THE INVENTION

A water-repellent skin is formed on a suitably dimensioned pad of open cell polyurethane foam cushioning material by spraying it at room temperature with a two-component spray-in-place flexible 100% solids elastomer polyurea system. The two components are a resin and isocyanate and the combination is applied through plural component high pressure impingement mix polyurethane dispensing equipment. The two components which are sprayed on the cushioning pad are the same as the components which are sprayed on the interior of the shell as described hereinabove in the process of using a mold for producing the product. Since, according to the instant invention, the skin-forming components are sprayed directly onto the pad of cushioning material in situ and at room temperature to produce the water-repellent bonded skin, the benefit of the savings in time and labor alone is apparent. Also, the method can be used for almost any size or shape of cushion.

### BRIEF DESCRIPTION OF THE DRAWINGS

FIG. 1 is a simplified diagrammatic illustration of a method for producing a product of the instant invention; and

FIG. 2 is a cross-section of a product made in accordance with the method illustrated in FIG. 1.

### DESCRIPTION OF THE PREFERRED EMBODIMENTS

The details of the materials used to produce the water-repellent skin on the polyurethane foam cushioning material and the equipment used in the process and the specifications as to any properties of the component materials and the various equipment settings as necessary to produce the end product, to the extent that they are publicly disclosed, are available from BASF Corporation of Wyandotte, Mich. Applicant makes no claim to inventorship of the component materials or equipment or equipment settings, etc. used in this process for forming the skin on the cushioning material. Nor does applicant make any claim of inventorship or originality with respect to the cushioning material itself. For the purposes of this application for patent, applicant presumes that the component materials, the equipment, the equipment settings, etc. are not unique and are publicly known or available so do not require any detailed description.

For producing a skin bonded directly onto the surface of the open cell polyurethane foam, as described in a product data sheet of BASF Corporation a two-component spray-in-place system is used. The two components are a resin and isocyanate which are fed separately into a high-pressure spray-in-place plural component dispensing equipment. In the instant invention the resulting combination of the two components is sprayed at room temperature directly onto a surface of a cushion or pad of polyurethane open cell foam in situ to produce a fast cure smooth surface which is water-repellent after curing and adheres or bonds firmly directly to the surface of the polyurethane foam without the need for any intervening adhesive layer.

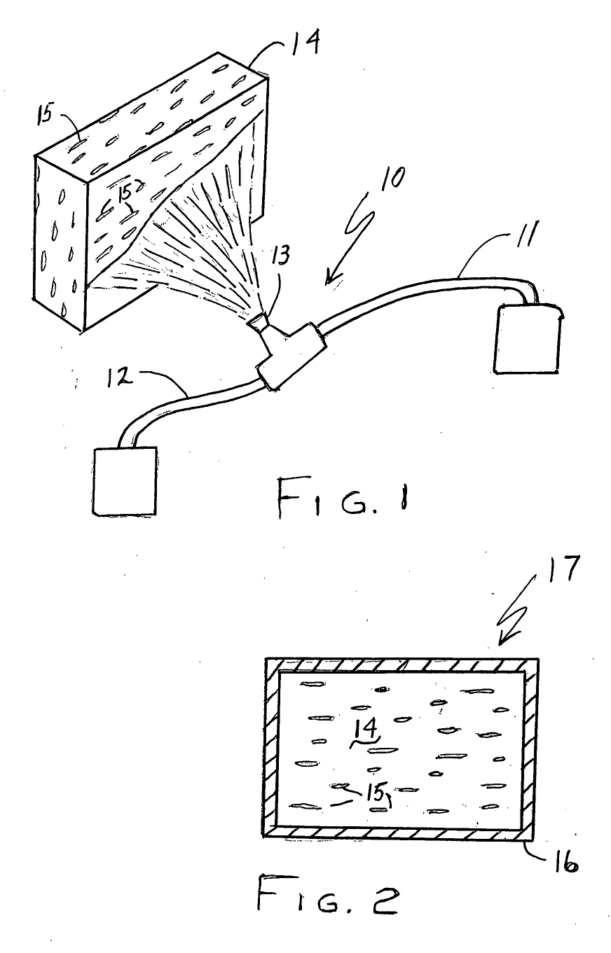

As illustrated diagrammatically in FIG. 1, the resin may be inserted into a suitable piece of spraying equipment 10 through an input conduit 11 and isocyanate is inserted separately via conduit 12. It is not clear whether the two components are mixed together within a chamber of the spraying equipment 10 or are ejected separately out of the nozzle 13 and combined in the air at the nozzle exit or upon impinging the surface of the cushioning material 14. In any event, the combination is sprayed onto a block 14 of polyurethane open cell foam cushioning material. The open cells as they appear on the surface of the cushioning material 14 are identified by reference numerals 15.

After the two-component material has been sprayed onto the surface of the cushioning pad 14 and suitable curing time has passed, as illustrated in cross-section, FIG. 2, the coated cushion 17 comprises a block or pad of material 14 with a thin layer 16 covering the exposed surfaces to which the spray is applied. As illustrated in FIG. 2, the cells 15 at the skin covered surfaces are no longer “open cells” since the cell openings are closed off by the skin 16 which is tightly adhered or bonded directly onto the exposed sprayed surfaces of cushioning pad 14.

In some cases it is not necessary to coat all the surfaces of the cushioning pad 14. For example, a boat seat pad usually rests on a supporting plate so the underside of the pad may not have to be covered with the coating skin. As illustrated in FIG. 2, the sprayed-on material spans the gaps in the open cell foam so closes off the open cells or pores of the foam to make the coated surface waterproof. Typically a suitable polyurethane open cell foam block used for a cushion on a seat may have a density in the range of about 1.6 to 2.8 pounds per cubic foot and may have compression or ILD in the range of about 40-120. Typically the waterproof skin may have a thickness in the range of about 0.030 to 0.040 inch.

According to the specification sheet the water-repellent coating formed in the manner described may not be light-fast and so over a period of time it might deteriorate when exposed to light and lose its water-repellent characteristic. For this purpose and for cosmetic reasons, the coated pad is usually covered with some type of cosmetically-pleasing vinyl or other suitable covering.

A significant benefit achieved by this invention is that a variety of shapes and sizes of blocks of cushioning material having a variety of contours for a variety of styles of seats or the like can be made with a water-repellent skin without the need of different molds for each style, shape or size.

I claim:

1. A pad of open-cell foam cushioning material having a thin water-repellent skin formed on a surface of said pad by spraying a combination of resin and isocyanate directly onto the surface of the pad to bond the skin directly onto the pad surface with no intervening adhesive layer.

2. A pad of open-cell foam cushioning material as described in claim 1 wherein the formed skin spans the open cell gaps on the covered surface.

3. A pad of open-cell foam cushioning material as described in claim 1 wherein the combination of components is sprayed on a surface of the pad in situ.

4. The process of making a cushioning pad, comprising the steps of:
a) providing a pad of open-cell foam cushioning material; then
b) spraying a combination of resin and isocyanate directly onto a surface of the pad to form a thin water-repellent skin on said surface bonded directly onto said surface with no intervening layer.

5. The process as described in claim 4 wherein the combination of components is sprayed on the pad in situ.

6. The process as described in claim 4 wherein the sprayed combination closes off any open cells on the sprayed surface.
